(19) United States
(12) Patent Application Publication
(10) Pub. No.: US 2018/0263655 A1
(43) Pub. Date: Sep. 20, 2018

(54) GUIDANCE DEVICE FOR ULTRASONOGRAPHY GUIDED DEVICE PLACEMENT

(71) Applicant: HELSE STAVANGER HF, Stavanger (NO)

(72) Inventors: Lars FJELLAND, Randaberg (NO); Sumit ROY, Hafrsfjord (NO); Nils Petter OVELAND, Stavanger (NO)

(21) Appl. No.: 15/758,404

(22) PCT Filed: Sep. 5, 2016
(86) PCT No.: PCT/EP2016/070826

§ 371 Date: Mar. 8, 2018

(30) Foreign Application Priority Data
Sep. 8, 2015 (SE) 1551145-4

(51) Int. Cl. A61B 17/34; A61B 8/08; A61B 8/00; A61B 46/10; A61B 50/30

(52) U.S. Cl. CPC A61B 17/3403; A61B 8/0841; A61B 8/0891; A61B 8/4209; A61B 8/4422; A61B 2050/0053; A61B 50/3001; A61B 8/4455; A61B 2017/3407; A61B 2017/3413; A61B 46/10

(57) ABSTRACT

A guidance device for ultrasonographic guidance of percutaneous placement of an intravascular introducer in a subject adapted for placement against the body of the subject essentially at a desired percutaneous insertion site is disclosed. A directing member is oriented such that the trocar or introducer received in the directing member provides for insertion of the trocar or introducer along a first axis. The guidance device is adapted to receive and releasably hold an ultrasonographic imaging probe such that a first axis of the directing member is positioned in a plane that is essentially perpendicular to and transversely crossing the imaging plane, and the directing member is coupled to the guidance device via an attachment member such that the directing member is configured to be movable only in the plane that is essentially perpendicular to the imaging plane.

### FIELD OF THE INVENTION

The present invention relates to a guidance device for ultrasonography guided placement of a medical device, and a guidance assembly including such a guidance device, according to the preamble of the independent claim.

### BACKGROUND OF THE INVENTION

Trauma is among the most common causes of mortality worldwide, accounting for about a tenth of the deaths occurring every year. In almost half the cases, loss of life after trauma can be ascribed to uncontrolled bleeding. Both blunt and penetrating injuries can cause serious internal bleeding, that not infrequently may be difficult to detect, and very often requires a major surgical procedure to stop. Therefore, even today, the immediate management of a severely bleeding victim of trauma, whether for example on the battlefield or at the site of a traffic accident, represents a major challenge. All that can be presently offered is intravenous infusion of fluids, external compression devises such as pelvic binders, inflatable trousers and tourniquets (abdominal, junctional and for extremities), followed by very rapid transfer to the nearest trauma hospital. As the latter is frequently not possible even with access to the best logistics, not unsurprising a large number of trauma victims around the world simply bleed to death. A large majority of these would probably escape this fate if the bleeding could be halted before the patient is moved from the site of trauma.

One technique for lessening or stopping excessive bleeding is resuscitative endovascular balloon occlusion of a blood vessel, commonly of the aorta or downstream of the aorta. This procedure is commonly abbreviated REBOA (“Resuscitative Endovascular Balloon Occlusion of the Aorta”). A balloon catheter is inserted one of the femoral arteries in the groin of a subject, and threaded up to the aortic bifurcation. Here the balloon is inflated to stop blood flow to the pelvic area. In many cases this is sufficient to avoid a catastrophic fall in blood pressure and restore central perfusion to vital organs such as the heart and brain, and to allow enough time for transport to a hospital. However, if the hemodynamic response is not satisfactory, the balloon can be deflated, repositioned and re-inflated higher up in the aorta. Hereby, also the blood supply to the kidneys and the gastrointestinal tract, via the visceral arteries, is hindered; further stopping any excessive bleeding out, and allowing blood to be concentrated to the brain and heart. Thereafter the patient can be transported to medical facilities for surgical measures.

The biological principles underlying REBOA are very well known. Severe bleeding leads to rapid reduction in circulatory blood volume. Loss of over 30-40% of the blood volume leads to circulatory collapse and death. Bleeding of this magnitude unfortunately cannot be compensated for by intravenous infusion of fluids. REBOA on the contrary offers an elegant therapeutic solution. Since the aorta is the sole source of blood to the body, halting aortic blood flow upstream of a bleeding artery leads to immediate reduction of flow in the latter, to the extent that bleeding ceases or is reduced to a negligible level. And as a bonus, the blood remaining in circulation is redistributed to more vital organs such as the heart, the brain and the kidneys.

As an example, a retrospective analysis of data in the UK Joint Theatre Trauma Registry revealed that 20% of military personnel with severe combat injuries would have been candidates for REBOA (Morrison J J. Shock 2014; 41:388-93). With conventional treatment, 70% of them died, most of them before they could be transferred to a hospital.

REBOA was first described in 1954 by an American military surgeon treating battle casualties in the Korean War (Hughes C W. Surgery 1954; 36:65-8). Since then a number of articles have appeared in literature focusing on use of the procedure during aortic surgery (Hesse F G. Ann Surg 1962; 155:320-2. Howard E R. BMJ 1971; 3(5767):161), upper abdominal surgery (Miura F. J Gastrointest Surg 2006; 10:519-22), and for treating patients with severe bleeding from the uterus (Harma M. Obstet Gynaecol 2004; 44:170-1), as well as after pelvic injuries (Martinelli T. J Trauma 2010; 68:942-8).

Many documents describe different types of balloon catheters for occluding vessels and for use in various other procedures. International patent application WO 2011/133736 describes an endovascular aortic occlusion system comprising a balloon catheter for occluding the thoracic aorta. U.S. Pat. Nos. 5,334,142 and 5,437,633 both disclose similar devices comprising balloon catheters for occluding the aorta during CPR, and further comprising means for infusing oxygenated fluid into the aorta above the occluded balloon.

Despite its obvious advantages, REBOA has unfortunately found very little use in general, and in pre-hospital settings, REBOA is practically unknown. The critical step in the procedure is gaining access to the artery in the groin for placement of a catheter or introducer, a procedure which requires experience even in controlled environments. In addition, in trauma situations, where the arterial pulse is weak or cannot be found, due to excessive bleeding, it becomes even more difficult.

Ultrasonography is commonly used for guiding puncture and introducer placement in blood vessels. Notably, using ultrasonography guided arterial puncture successfully requires a substantial amount of training to be performed even in the best of circumstances. The commonly used technique to safely access e.g. blood vessel is called the Seldinger technique. First a trocar, i.e. a hollow needle with a sharp tip, is inserted at an angle through the skin and underlying tissue into a blood vessel. A guidewire is then threaded through the hollow needle, and the needle is thereafter withdrawn, leaving the guidewire inserted into the blood vessel. Depending on the following procedure, an introducer, a catheter or other hollow device can be threaded safely over the guidewire, to access the interior of the blood vessel.

Needle guides are sometimes used to make the procedure easier. US Patent Application 2002/0026117 shows a medical probe for ultrasound guided insertion of a medical device. US Patent Application 2012/0165679 and WO 2010/019795, each disclose a device for orthogonal ultrasonography guided puncture. However, these devices do not provide any means of containing the sterile filed around the access site, making them unsuitable for use outside the hospital setting. Further, they do not take into account the fact that commercially available ultrasonography probes, even from the same vendor, often have different external shapes.

REBOA can be performed with commercially available products (Stannard A. J Trauma 2011; 71:1869-1872). Nonetheless these products were designed to be used in a suitably large sterile operative field, by doctors with the training and experience to perform catheter-based, i.e. endovascular, procedures. Out in the field or in an emergency room, space is at a premium, and a sterile environment can be hard, if not impossible, to achieve, and the personnel may have little endovascular experience.

Thus, there is a need for a solution allowing an easier and safer way of gaining access to large blood vessels via an introducer for procedures such as performing REBOA, especially outside medical facilities, such as at accident sites or in military settings.

### SUMMARY OF THE INVENTION

An object of the present invention is to provide a guidance device providing a rapid non-surgical access to the vascular system in a subject.

Another object is to provide a guidance device for vascular access which can assist and guide the user in the insertion procedure, thus providing safer and more reliable results. A further object is to provide a guidance assembly comprising a guidance device that allows a procedure requiring vascular access to be performed in hygienically challenging environments.

The above-mentioned objects are achieved by the present invention according to the independent claim. Preferred embodiments are set forth in the dependent claims.

A guidance device for ultrasonographic guidance of percutaneous placement of an intravascular introducer in a subject adapted for placement against the body of the subject essentially at a desired percutaneous insertion site is disclosed. The guidance device comprises a directing member adapted to receive a trocar or an introducer for percutaneous insertion of the trocar or introducer into a blood vessel of the subject. The directing member is oriented such that said trocar or introducer received in the directing member provides for insertion of the trocar or introducer along a first axis. The guidance device is adapted to receive and releasably hold an ultrasonographic imaging probe to obtain an image having an imaging plane. Further, the guidance device is arranged to hold the ultrasonographic imaging probe such that the first axis of the directing member is positioned in a plane that is essentially perpendicular to and transversely crossing the imaging plane, and the directing member is coupled to the guidance device via an attachment member such that the directing member is configured to be movable only in the plane that is essentially perpendicular to the imaging plane.

A guidance assembly comprising such a guidance device is also provided, in which the guidance assembly may also comprise an occlusion device for occluding a blood vessel of a subject, as well as a trocar and an introducer, and is packaged in a suitable container.

### SHORT DESCRIPTION OF THE APPENDED DRAWINGS

FIG. 1 shows two examples of a guidance device for ultrasonography guided catheter placement.

FIG. 2 shows a guidance device placed in the intended area of use on a body.

FIGS. 3a-3c illustrate the relationship between ultrasound imaging plane and a guidance device.

FIG. 4 shows an occlusion device for occluding a blood vessel.

FIG. 5 shows a guidance assembly for ultrasonography guided device placement.

FIG. 6 shows another guidance assembly for ultrasonography guided device placement.

FIGS. 7a-7c illustrate the use of a guidance assembly for ultrasonography guided device placement.

FIGS. 8a-8c show different perspective views of a guidance device.

### DETAILED DESCRIPTION OF THE INVENTION

Physicians and paramedics who deal with casualties of accidents, whether in a civilian environment, or on the battlefield, as well as acute care specialists and any medical personnel who deal with patients with uncontrollable bleeding trauma, including trauma and vascular surgeons, gynecologists, interventional radiologists and anaesthesiologists, would benefit from an improved device for guidance of a trocar or introducer into a major blood vessel. Such a device for providing vascular access should be able to assist and guide the user in the insertion procedure for quickly and safely performing procedures such as REBOA, where rapidly halting blood flow in the aorta is essential. Also in other procedures, e.g. catheterization for different purposes, such as dialysis, cooling, cardiac monitoring, placement of medical devices inside the vascular system and various other procedures, a device for guiding the insertion is of great value, especially in cases where time is essential and/or the user is less experienced in accessing a desired vessel.

In the illustrative example of performing REBOA, one challenge of the procedure in non-hospital or emergency settings is having personnel on hand who are trained in performing REBOA, which is a relatively complicated and invasive procedure. One of the critical steps of performing REBOA is gaining access to the femoral artery in the groin for placement of an occlusion device in the aorta. Under the best of circumstances, there is always a risk of incorrect puncture, uncontrolled bleeding, or simply not placing the introducer for access in the correct vessel. One common problem is accidently placing the puncture and introducer in the femoral vein, instead of the femoral artery, as the two run alongside each other in the groin.

In addition, accessing the femoral artery in the groin area can be especially difficult in a trauma patient who has sustained any number of injuries, resulting in bleeding, broken bones and injured internal organs. A common procedure in e.g. traffic accidents is to place a so-called “pelvic binder” around the pelvis of the patient, to stabilize any fractures in the pelvic bones. The pelvic binder is a belt that is placed around the patient's pelvis and applies an external circumferential pressure to stabilize any pelvic fractures. This will be described in more detail below. Thus, accessing the groin area, and performing a procedure that includes entering the large femoral artery is complicated in a trauma situation, and can be very stressful for a clinician to perform.

An additional challenge is to have all the necessary components within reach, and a further challenge is keeping such components and the area of use sterile, all while working in a dirty and/or contaminated as well as stressful environment. As an example, to perform REBOA, several components are needed, such as an occlusion device, an ultrasound probe and device for viewing the entry site, a long guide wire for introducing the occlusion device, an introducer, a needle or other device for penetrating the skin, means for inflating the balloon etc. All the components need to be handled and used in a sterile manner to prevent contamination and infection.

Moreover, as is well known, massive internal bleeding, with consequent deterioration of cardiovascular status, can occur due to any of a number of conditions not related to trauma. In the absence of a satisfactory alternative, urgent surgery is often resorted to in these patients. Not surprisingly, the outcome is not infrequently far from satisfactory, because unstable cardiovascular parameters make these patients particularly ill-suited for surgical intervention, especially including an anesthesia procedure. Routine preliminary REBOA in these patients has the potential to dramatically improve their prognosis. Thus, also in such hospital settings, REBOA could be used to save lives if there was a way of carrying out the procedure that was easier to learn and perform.

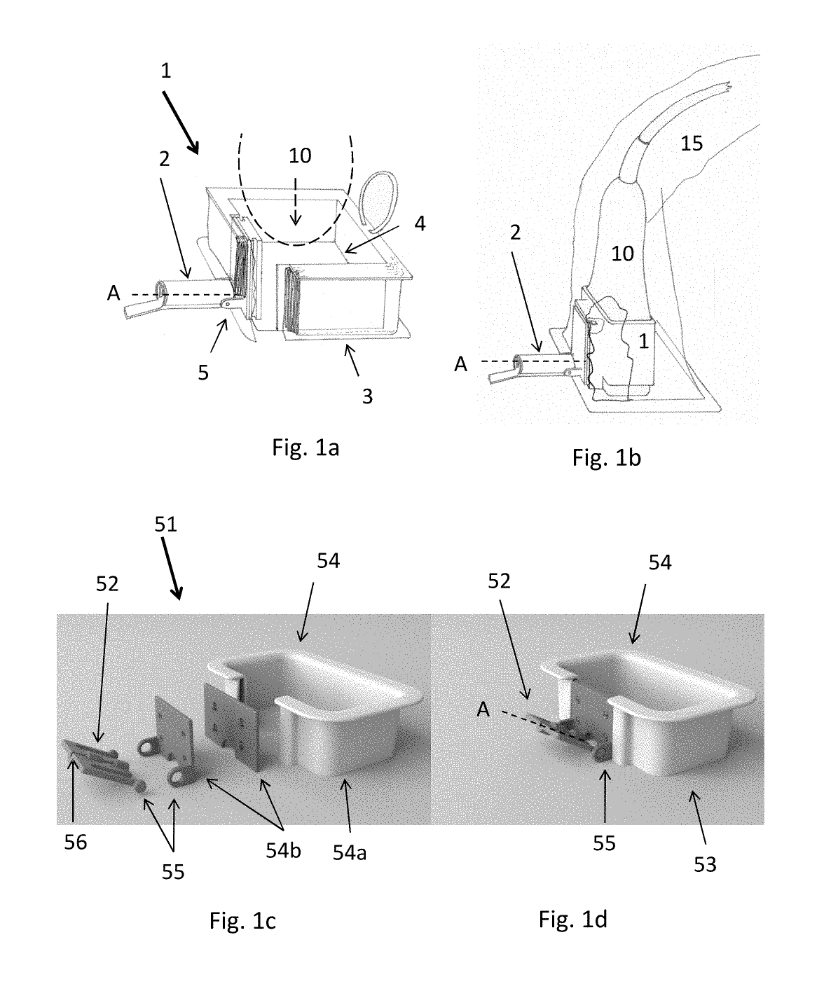

A guidance device is presented here, which offers such a solution. In FIG. 1a, a guidance device 1 for ultrasonographic guidance of percutaneous placement of an intravascular introducer in a subject is shown in a perspective side view. The guidance device 1 comprises a directing member 2 for a trocar or introducer for percutaneous insertion into a blood vessel of a subject, wherein the guidance device and the directing member are arranged to assist a user in percutaneous insertion of said trocar or introducer into said subject.

In the present disclosure, the relative term “proximal” refers to a direction or part of a device or component being closest to a user, and thus “distal” refers to a direction or part of a device being farther away from a user. This is particularly applicable to e.g. a trocar or an introducer for accessing a blood vessel percutaneously or to a catheter device, such as that used in the REBOA procedure, wherein the distal end is inserted first into a patient, and, during use, is the end which reaches farthest into the vascular system. The proximal part of a trocar, an introducer or a catheter is that which normally remains outside the patient.

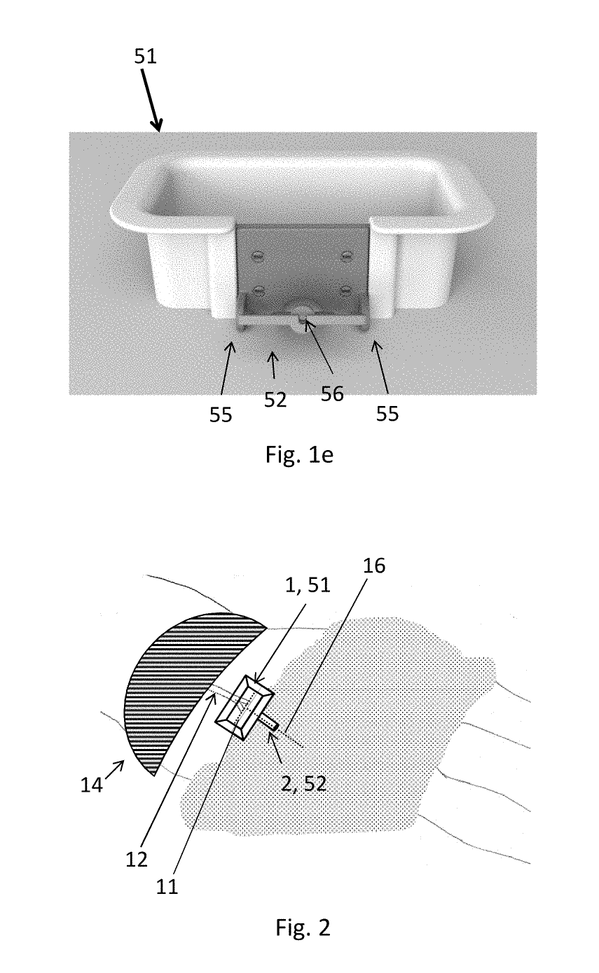

Herein is also referred to other relative directions which will become clear from the description. In general, when vascular access is desired via the femoral artery, such as in a REBOA procedure or other procedures, it is assumed a patient is lying on his/her back on an essentially horizontal surface, and the guidance device is placed in one of the groins of the patient, as illustrated in FIG. 2, which shows a schematic top view (generally from above the patient) of a guidance device, and how it is intended to be placed in the groin area. It should however be noted, that the guidance device according to the present disclosure may also be adapted for use in accessing other major vessels, such as the radial artery or the jugular vein. Such examples will be described in detail further below.

Thus, for femoral vascular access, the guidance device 1 is placed in the groin area with the backside 3 against the skin of the patient, and the directing member 2 extending towards the foot of the subject or patient. This is illustrated in FIG. 2. Preferably, the guidance device is secured to the patient. This can be done by simply taping or strapping down the guidance device on the upper thigh of the patient. The guidance device can also be provided with attachment means such as straps, such as Velcro-straps or self-adhesive straps, for attachment to the patient. Notably, in trauma situations where REBOA is indicated as a suitable procedure, the patient often has pelvic bone injuries. Thus, a common procedure in such situations is to secure a so-called “pelvic binder” around the pelvis of the patient, to stabilize the pelvic region. A pelvic binder 14 is a wide belt, and covers the pelvis of the patient, which is schematically indicated in FIG. 2. Thus, access to the groin area is limited. The present guidance device 1 is adapted to this situation, in that it is small but adapted to fit over a suitable entry site for accessing the femoral artery.

Thus, the guidance device 1 is adapted for placement against the body of the subject essentially at a desired percutaneous insertion site for a trocar or introducer. This will be explained further below.

The guidance device 1 is also adapted to receive and releasably hold a probe 10 for ultrasonographic imaging during insertion of trocar or an introducer into a blood vessel, as is shown in FIG. 1b. For comparative purposes, in FIG. 1a, the probe 10 and its intended insertion direction into the guidance device is shown in dotted lines. Thus the guidance device has a cavity 4 formed by a surrounding bracket structure for receiving a distal end of an ultrasound probe 10, where the cavity 4 is adapted to accommodate a number of different available ultrasound probes 10. Notably, if needed, adaption to different ultrasound probes may be achieved by using an adapter, as will be explained in detail further below. The side of the guidance device facing the patient, i.e. at the bottom of the cavity, is sonolucent, i.e. allows ultrasonographic imaging of underlying structures. As an alternative, only a central part of the bottom of the cavity is sonolucent, such that only structures which are in the middle of the field will be visible. Restricting the field of view allows for optimal placement of a probe 10, as well as keeping the probe in an optimal position during the following procedures, as will become clear from the details described below of the relationship between the resulting image and the angle of penetration directed by the directing member.

The bottom and/or sides of the cavity 4, i.e. on the inside of the cavity, can optionally be provided with a sonolucent adhesive for improved hold of a probe in the cavity.

Common ultrasonography probes have a generally oblong distal end, wherein the longer central axis of the oblong shape defines the imaging plane of the resulting image. The ultrasonographic probe has a leading surface, wherein the leading (distal) surface does not appreciably hinder the passage of sound waves emitted by the probe, i.e. the probe has a distal sonolucent surface. Thus, an important feature of the guidance device 1 is to align the received probe 10, regardless of type, such that an imaging plane 11 is obtained in a direction transverse to underlying major blood vessels of a groin area. In the top view of FIG. 2, the resulting imaging plane 11 of a probe inserted into the guidance device 1 is indicated with a dotted line. The femoral artery 12 is also shown in dotted line. The present inventors have realized that using a transverse cross-section for ultrasonography imaging of the major femoral blood vessel provides for a technique in guiding puncture of the desired vessel which is easier to learn and better suited especially for less experienced users, such as first responders at an accident site. The advantages will be further described below.

The guidance device may also comprise a probe cover 15 as shown in FIG. 1b. The probe cover 15 is a generally tube-shaped transparent cover intended to isolate the ultrasonography probe from the sterile field surrounding the guidance device. The distal end of the probe cover 15 is attached to the bracket surrounding the cavity 4 such that probe can be inserted into the cavity 4 without contaminating the sterile field. The directing member 2 is arranged outside the probe cover 15.

Using the guidance device 1 as described above helps align the path of an introducer with a suitable entry path into a femoral artery 12 of a subject. Hence, the guidance device 1 is arranged to hold the probe 10 for ultrasonographic imaging such that the longitudinal axis A of the directing member 2 is positioned in a plane that is essentially perpendicular to the imaging plane 11. Further, as can be seen in FIG. 2, the directing member thereby also is positioned in a second plane 16 that is essentially aligned with the femoral artery 12 when the device is placed in position on a patient. Further, the directing member 2 is configured to be movable only in the plane 16 that is essentially perpendicular to the imaging plane, i.e. up and down to adjust the depth angle of penetration, as will be explained further below. This may be achieved by coupling the directing member 2 to the bracket of the guidance device via an attachment member 5, such as a hinge or similar attachment.

The directing member 2 may be a generally tubular structure, as shown in e.g. FIGS. 1a and 1 b. As an alternative, a directing member can comprise an open structure with one or several grooves or pre-formed slots in a suitable surface (not shown), wherein the grooves or slots are adapted to accommodate a needle or introducer and direct insertion in the intended direction. One example is a vertically oriented plate with multiple canals or grooves in a side surface, which is attached to the outside of the guidance device. Such an open structure can similarly be coupled to the bracket of the guidance device via an attachment member, e.g. a hinge or similar structure, or comprise a fixed or detachable structure on the outside of the guidance member. Each groove or slot will define a longitudinal axis of the directing member and be positioned in a plane that is essentially perpendicular to the imaging plane. Each groove or track may be marked with a visual indicator of the depth of penetration when using such groove or slot.

Another exemplary directing member 51 is shown in FIGS. 1c, 1d and 1 e. FIG. 1c shows an exploded view of the guidance device 51, and FIG. 1d shows the assembled guidance device 51. In these figures, the directing member is in the form of a needle holder 52. The bracket 54 forms a cavity adapted to hold the distal end of an ultrasonography probe, and may comprise, as illustrated in the figures, a surrounding bracket part 54a and one or two needle holder attachment plate(s) 54b. This arrangement is advantageous for manufacturing reasons, as each piece may be made separately and thereafter assembled. However, the bracket 54 may alternatively be a single unitary piece. A probe cover (not shown in FIGS. 1c-1e) may also be provided, similar to the probe cover shown in FIG. 1b, to ensure the sterile field as described above.

The directing member or needle holder 52 is adapted to be removable from the bracket structure 54, but still be sufficiently attached via hinge 55 during use, i.e. be able to hold the needle or introducer in the desired position and only be movable in the desired direction, as described previously. In the illustrated example, the needle holder 52 is provided with hinge elements 55 adapted to be snapped into hinge elements 55 in the needle holder attachment plate(s) 54b. Optionally, after use, with a needle still arranged in the directing member, the directing member may be loosened from the bracket structure, such that the bracket, a probe cover and the ultrasonography probe may be removed from the patient.

The needle holder 52 is further provided with a through-going hole 56 adapted to receive a needle or introducer. In FIG. 1e is shown a view in a direction looking along longitudinal axis A. In the illustrated example, this through-going hole 56 is formed by a combination of grooves and enclosing structures, however other arrangements using open or closed slots or hole(s) may be used.

Other hinge arrangements and needle holder configuration arrangements are possible, as long as the directing member 52 is adapted to hold an inserted needle or introducer along a longitudinal axis A of the directing member 52, wherein the needle will be positioned in a plane that is essentially perpendicular to the imaging plane, as described previously.

Notably, even though the general structure of the guidance device and especially the cavity 4 and bracket 54 will fit most common ultrasonography probes, the guidance device can further be provided with one or several probe adapters, i.e. structures that fit into the cavity and have an adapted upper surface to accommodate specific shapes of the distal leading edges of a probe. Such adapters are preferably held in the cavity by friction, a snap-lock or by an adhesive.

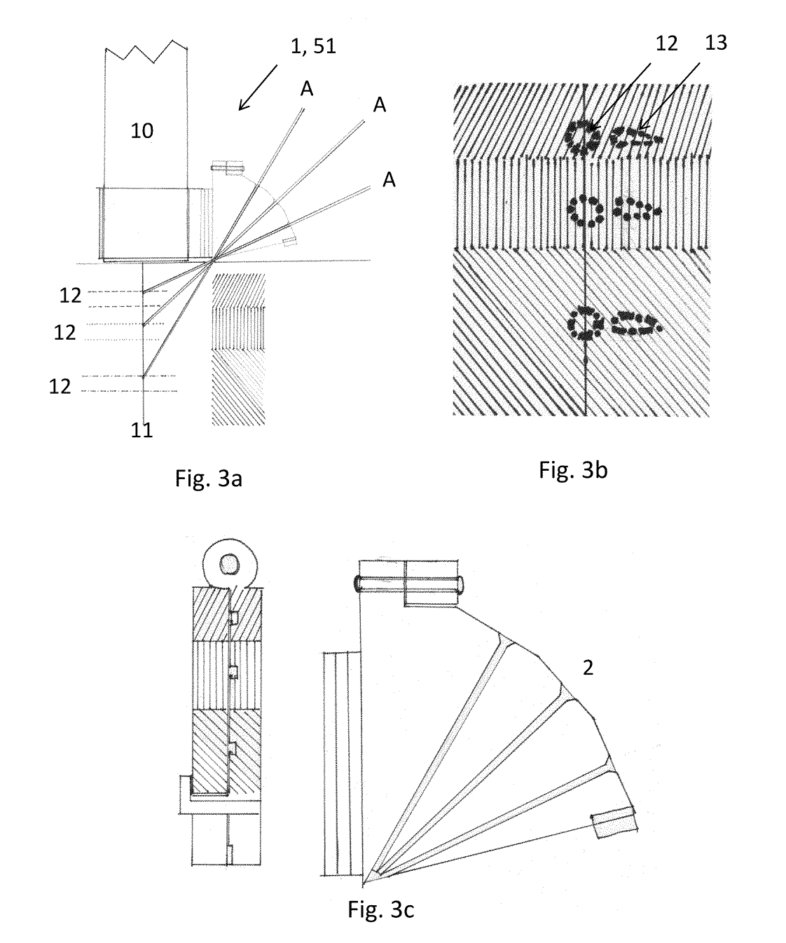

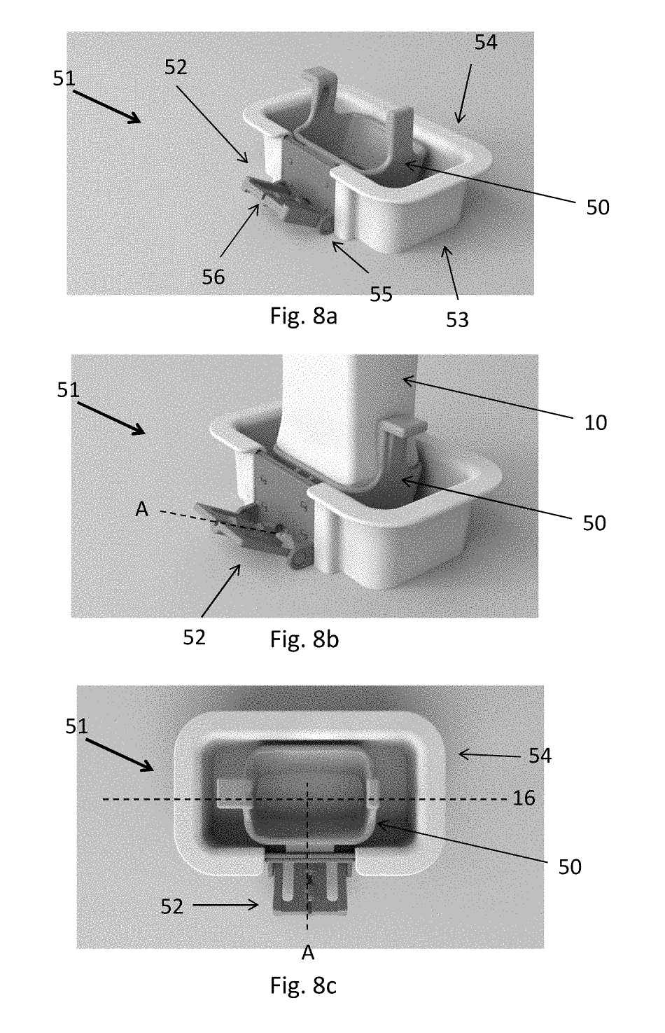

In FIG. 8, a further guidance device is shown. In this aspect, the guidance device further comprises a probe adapter 50 for the distal end of the ultrasonography probe. The probe adapter 50 is configured to be arranged in the bracket 54 and receive a distal end of the ultrasonographic imaging probe 10, as shown in FIG. 8b. The adapter is configured to hold the probe 10 in a position such that the first axis A of the directing member 52 is positioned in the second plane 16 (as shown in FIGS. 2 and 3) that is essentially perpendicular to and transversely crossing the first plane 11, i.e. imaging plane 11 of the ultrasonography probe. In FIG. 8b the guidance device 51 with adapter 50 is shown in a side view and in FIG. 8c the guidance device is shown from above, i.e. along the direction of inserting the ultrasonography probe 10 into the adapter 50. The ultrasonography probe 10 is not shown in FIG. 8c for illustrative purposes. Here it is clear that using an adapter 50 together with the guidance device as described will ensure that the probe 10 is properly positioned such that the directing member 52 will guide the needle or introducer along longitudinal axis A as described above.

In FIGS. 8a-8c a single adapter 50, adapted to the specific probe 10, is shown. However, preferably a number of adapters, each configured to fit different commercially available ultrasonography probes, is provided with the guidance device 1, 51, such that a user may easily chose a fitting adapter to whatever ultrasonography probe that might be available in a particular situation.

For femoral access to a blood vessel, the guidance device 1, 51 may be configured to be placed by a user in the groin of a subject at e.g. an accident site. The outside of the guidance device 1, 51, especially the backside 3, 53, i.e. the side adapted to be placed on the subject, can be shaped such that it corresponds to the general shape of a human or mammalian groin area. A guidance device 1, 51 may be adapted to be placed at or slightly below the fold between the upper thigh and the abdomen, and generally resting against the upper thigh.

A guidance device 1, 51 as disclosed may also be adapted for blood vessel access at the wrist of a patient, e.g. the radial artery, or at the neck region, e.g. the jugular vein. In such a case, especially the backside 3, 53, i.e. the side adapted to be placed on the subject, can be shaped such that it corresponds to the general shape of a human or mammalian wrist or neck.

As an alternative the guidance device 1, 51 can be mounted on a support or plate. Preferably, such a support has a generally rounded shape, to adapt to the shape of an upper thigh, wrist or neck region.

The two largest vessels in the groin area are the femoral artery 12 and the femoral vein 13. In FIGS. 3a and 3b, three exemplary different possible depths of these two vessels under the skin are shown in relation to corresponding longitudinal axis A of a directing member. As seen in FIG. 3b, the two major vessels will be displayed next to each other as circular black structures on the imaging plane in when using the guidance device.

FIG. 3a shows a side view of a guidance device 1, 51, with an ultrasonography probe 10 inserted into it. Below the guidance device 1 is shown a vertical line, indicating the imaging plane 11 of the ultrasonography probe. In FIG. 3b, a corresponding resulting image is shown. The guidance device is placed in the groin area of the patient, and in FIG. 3a is seen a plane defined by the directing member 2, 52, having a longitudinal axis A, and the underlying the femoral artery 12. It is to be noted that there is normally only one femoral artery 12 in each groin of a patient, however, in FIGS. 2a and 3b, three different exemplary depths of the femoral artery 12 is shown.

As is illustrated in FIG. 2, due to the configuration of the guidance device 1, 51 and the directing member 2, 52 for a needle or an introducer, when the guidance device 1, 51 is placed in the groin area of a subject, it provides a preset placement position for an ultrasound probe 10. Further, it also aligns the directing member 2, 52 towards a suitable entry point into the femoral artery 12. Due to the configuration of the guidance device, the projected entry direction of a needle or an introducer, along a longitudinal axis A, will transversely cross the imaging plane 11 of the ultrasound probe, i.e. the direction of introduction of the needle will be out-of-plane in relation to the imaging plane 11. This imaging plane 11 has the further advantage of being able to visualize both the femoral artery 12 and the femoral vein 13 in the same image, as illustrated in FIG. 3b. Thus, a user can ensure that the introducer is introduced into the femoral artery 12, and not the femoral vein 13 (or other vessels), as both can be visualized at the same time, in the same imaging plane.

The direction of the directing member is thus configured to be adjustable in a vertical plane that is perpendicular to the imaging plane 11. Preferably, the vertical plane will also bisect the imaging plane 11. As described previously, this can be accomplished by attaching the directing member 2, 52 with a hinge 5, 55 or similar connection means to the guidance device 1, 51, such that the directing member 2, 52 can be adjusted up or down, i.e. increasing or decreasing the angle of penetration into the underlying subject. This allows on-site adjustment due to individual patient body types, without compromising the guiding function of the guidance device.

As an alternative, a guidance device can comprise a directing member having one or several slots or grooves for inserting a needle or introducer in a fixed angle. One example is shown in FIG. 3c. Such fixed slots can be color-coded or marked to correspond to specified depths and/or regions of an ultrasound image.

When the guidance device is adapted for other regions than the groin and femoral artery access, the pivot point between the directing member 2, 52 and the bracket 4, 54 and/or the probe position may be adapted to the placement of the desired vessels.

The guidance device may be used in a REBOA procedure, for guiding an occlusion device smoothly, safely and securely into a percutaneous entry site into a femoral artery. It may also be used in any other procedure wherein vascular access is desired, as described above.

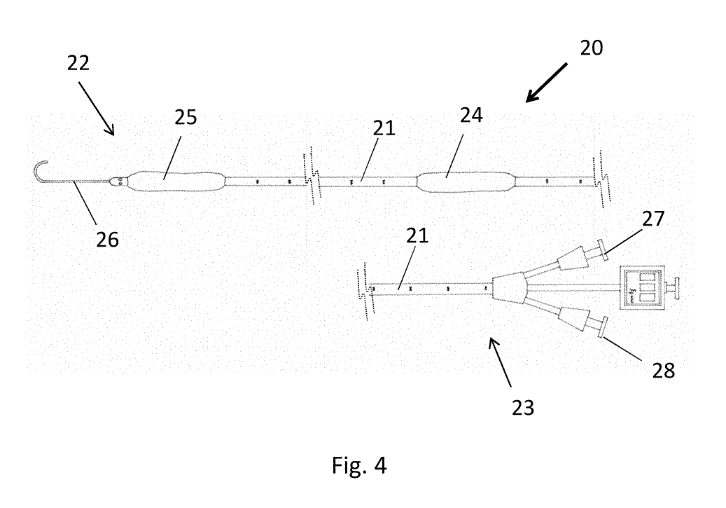

FIG. 4 shows an occlusion device 20 for occluding a blood vessel and for use with the present guidance device. The occlusion device 20 may comprise an elongated catheter 21 having a distal end 22 and a proximal end 23, where the catheter comprises at least a first balloon 24 adapted to occlude a blood vessel when in an expanded or inflated state.

The occlusion device can be adapted to be used together with a separate guide wire. However, preferably, the occlusion device can comprise a flexible, preferably J-shaped, guide wire part 26 forming a distal tip of the occlusion device 20. An occlusion device can thus comprise a J-shaped guide wire part 26 arranged distally of the catheter 21 and attached to the distal end of the catheter to extend distally therefrom and form a distal tip of the occlusion device. This allows the occlusion device to be safely advanced though the pelvic arteries to the aorta without the need for a long guide wire. Such an integrated guide wire also has the advantage of making it easier to keep the occlusion device and other components sterile, as no long guide wire needs to be handled outside the patient. In addition, one step in the procedure, i.e. having to first insert a guide wire and thereafter the occlusion device, can be eliminated.

An occlusion device 20 can also comprise a second balloon 25 adapted to occlude a blood vessel when in an expanded state. Preferably, the first and second balloons are arranged such that each can be expanded separately, e.g. via separate entry ports 27, 28 in the proximal end 23 of the occlusion device 20. The first balloon 24 is arranged such that it can be placed in the abdominal region of the aorta, and the second balloon 25 is arranged such that it will be located in the upper, thoracic, part of the aorta when the first balloon 24 is in the abdominal region of the aorta. The advantage of arranging two balloons in such a way on the occlusion device is that the first balloon can be inflated in the abdominal region, and if this procedure is not sufficient to stop bleeding to an acceptable level, the second balloon can be immediately inflated in the upper aorta. Thus, a step of deflating and repositioning a balloon is eliminated, saving valuable time.

An example of an occlusion device, or REBOA catheter, for use with the guidance device is described herein. However, this is only an example of the type of device which can be used. A REBOA catheter may be approximately 50 cm to 70 cm long, preferably approximately 60 cm long. It can comprise two occlusion balloons and a coaxial guidewire. The outer diameter of the catheter shaft may be 2.3-2.7 mm (7-8 Fr). The shaft may be reinforced to resist buckling in response to the strong axial forces on a balloon obstructing blood flow in the aorta.

If a separate guidewire is used, the guidewire may be approximately 70 cm long, and 0.89 mm (0.035 inches) in diameter. The leading end of the guidewire may be J-shaped (diameter J 7-10 mm), and protrude from the tip catheter, while the trailing end is reversibly secured to the catheter's hub. As alternative, an integrated distal guide wire tip may be used, as described above, with similar diameter and J-shapes tip. An integrated guide wire tip is preferably approximately 8-15 cm long.

The REBOA catheter may be a triple lumen catheter, and may comprise a coaxial inner relatively stiff cannula. The cannula prevents the catheter from buckling in response to the strong axial hemodynamic forces generated in the aorta when flow through it is halted.

The inflation channel for each balloon exits the catheter shaft in the form of a side-arm 27, 28 attached to the proximal end of the catheter. Each side are may be labelled e.g. “THORAX” and “ABDOMEN” and/or color-coded (e.g. red and blue or other contrasting colors or patterns) as appropriate, to enable the user to quickly identify the desired channel to connect to for inflation of a specified balloon. To inflate the balloons syringes prefilled with sterile saline may be used by attaching a syringe to the appropriate sideport 27, 28.

The catheter may also have on or more sideports for rapid infusion of fluids including blood or blood products. Thus, the catheter can be used both for occluding blood vessels, such as the aorta, and simultaneous infusion of e.g. blood, blood products or oxygenated fluid above the occluded vessel. Such infusion can thus at least partially replace the lost blood and improve oxygenation of the upper parts of the body, including vital organs such as the heart and brain.

The occlusion balloons can be independently inflated of the other, as indicated above. Thus flow in the can be halted at different levels in the aorta allowing bleeding from both the upper branches and the lower branches of the aorta to be treated without moving the catheter. As an example, the distal balloon 25 (thorax balloon) may be mounted approximately 2 cm from the leading, or distal, end of the catheter. The length of the balloon may be about 4 cm. The outer diameter of the catheter at the level of the balloon preferably does not exceed 3 mm (9 Fr). The proximal balloon 24, the abdomen balloon, may be mounted at a distance of 20 cm from the thorax balloon.

An occlusion device can further comprise sensors adapted to measure blood pressure, oxygen saturation, pH or other physiological parameters via the elongated catheter, for continuous monitoring of the procedure and the patient's health. A sensor may be mounted near the proximal end of the catheter or may be mounted at a distal end of the catheter. An integrated display is preferably provided at the proximal hub for continuous monitoring of the physiological parameter. Data from the sensor(s) may be transferred either through a cable inside the catheter or wirelessly to a suitable device able to display the information.

A guidance device as described above may be an integrated part of a guidance assembly for performing occlusion of a blood vessel. Such an assembly may be optimized for field use and trauma situations, and further may comprise an occlusion device for occluding a blood vessel of a subject, for example an occlusion device as described above. Moreover, the guidance assembly comprises at least a trocar and/or an introducer for percutaneous insertion of the occlusion device into a blood vessel of a subject.

In another aspect, a guidance device 1, 51 may be an integrated part of a guidance assembly for performing any procedure where a needle or trocar and optionally thereafter an introducer is percutaneously inserted into a blood vessel. Such an assembly may be optimized for field use and trauma situations.

The guidance assembly comprising at least a guidance device can be packaged in a suitable case or container. A non-limiting example is a robust, disposable, water-proof multifunctional case made from a preferably pathogen-proof, non-allergenic, sterilizeable material. The material is preferably also light-weight. The container should preferably be large enough to carry e.g. a REBOA catheter or other desired device and associated accessories, yet small enough to be comfortably transported by a paramedic, preferably hand carried.

An example of a guidance assembly comprises a case or container as described above, a guidance device 1, 51 as disclosed above, and one or several probe adapters 50 as described above. This makes it easy for a user to correctly place the guidance device directly at the desired entry site, chose a suitable probe adapter and helps direct and eases the insertion of a needle or introducer into the correct blood vessel as described previously.

Preferably, such an assembly comprises a probe cover to ensure sterility at the entry site. In addition, a guidance assembly may comprise side pieces or flaps, sterile drapes or other parts that are designed such that on opening the assembly, an adequately large sterile working area becomes available to the operator.

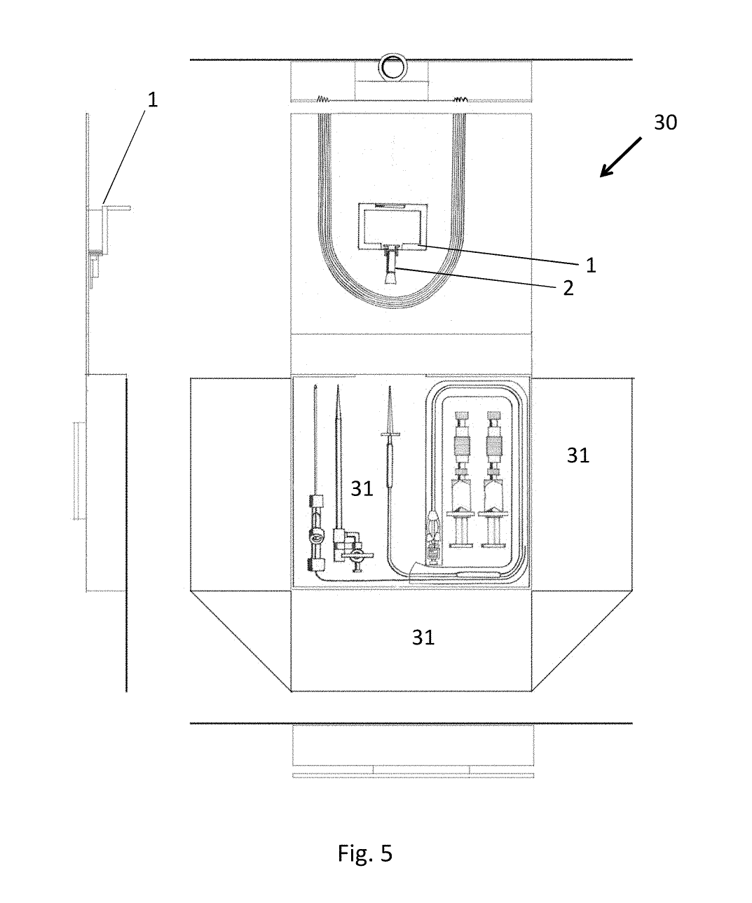

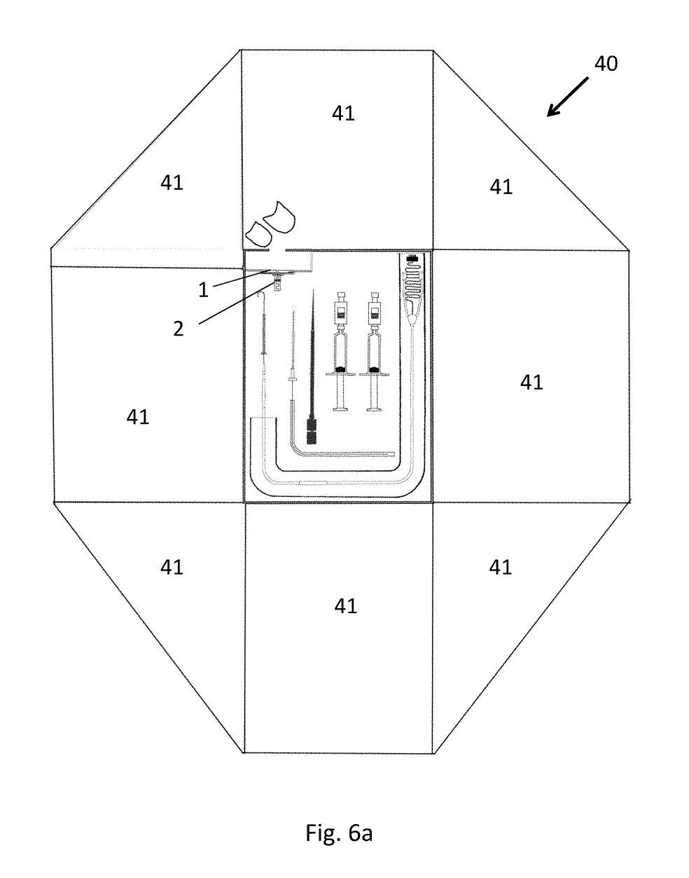

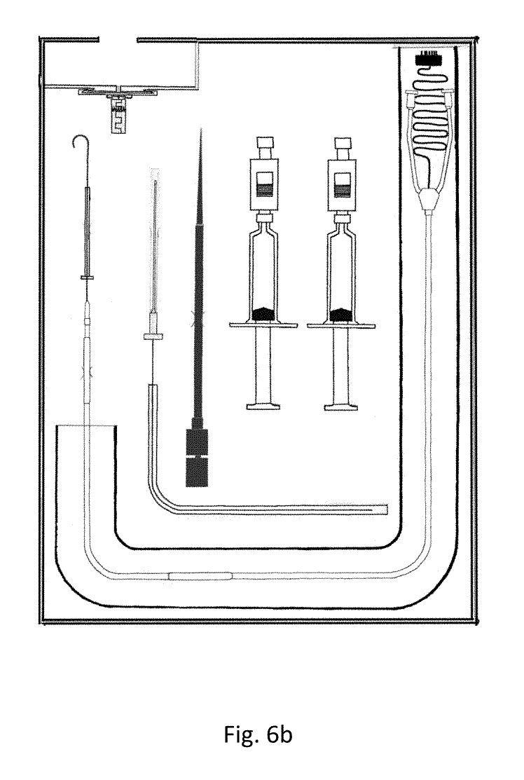

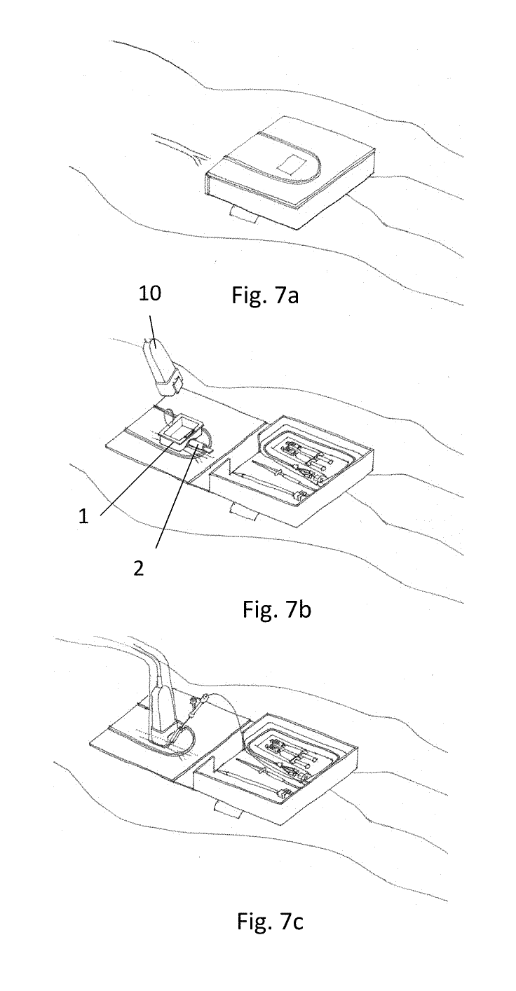

Another example of a guidance assembly is such an assembly adapted for a REBOA procedure, which is schematically illustrated in FIG. 5. A further example is shown in FIGS. 6a and 6b. The assembly 30,40 comprises an introducer and/or needle for puncture of the skin, a dilator to widen the entry hole, an occlusion device as previously described and preferably two-prefilled syringes for inflating the balloons. All components are releasably attached to the bottom of the case. Other accessories such as at least a pair of sterile gloves, sterile ultrasound gel and sterile wipes or sterile fluid can be provided in the assembly. The case 30, 40 is adapted to be opened and folded out to the illustrated configuration, directly on the patient's body. See FIG. 7a-7c for the use of such an assembly. Integrated in the assembly, at an upper part in FIG. 5, i.e. to be placed in the direction of the patient's head, is a guidance device 1 for ultrasonographic guided placement of a REBOA catheter as described above. The guidance device 1 is designed to be adaptable to an ultrasonographic probe irrespective of vendor as previously described.

The case may be provided with means to atraumatically secure it to the thigh of the patient, at the start of the procedure, regardless of the type of procedure. The attachment means can be any suitable attachment means, such as a belt or band comprising Velcro, fasteners, clasps and/or buttons. The case can be securely fixed to the patient's thigh irrespective of the size of the latter, such that all its contents are easily accessible to the operator. An example of a guidance assembly and how it is placed in relation to a patient is shown in FIGS. 7a-7b.

The case may so designed that on opening it, an adequately large sterile working area becomes available to the operator. Additional flaps 31, 41 can be provided, such that on folding out the flaps from the case, a larger sterile working area becomes available. Further, as described for the guidance device 1, 51 above, on securing the case to the patient and folding out the contents, the guidance device 1, 51 will be in position to guide the entry into the femoral artery, as described above and seen in FIG. 7b. Hence, once the case is in place on the thigh, the guidance device 1, 51 will be automatically located at the groin for arterial puncture as described above. Moreover, the guidance assembly can preferably comprise also at least a pair of sterile gloves, sterile ultrasound gel and sterile wipes or sterile fluid to make the procedure as safe in terms of contamination as possible.

The guidance device 1, 51 may be attached to the case such that it can be rotated and/or moved in all axes in the horizontal plane defined by the backside of the guidance device, to allow it to be optimally placed with relation to the artery to be punctured. A locking mechanism is preferably provided, such that after such adjustment, the guidance device may be locked in the desired position.

As described above, contrary to other ultrasound products on the market, the guidance device 1, 51 allows arterial puncture using “out-of-plane” technique. Based on the experience of the inventors, this technique is far easier to learn than its conventional “in-plane” counterpart, and significantly reduces the risk of inadvertent catheter placement in the large vein which runs parallel to the femoral artery in the groin.

As described above, the needle trajectory can be changed depending on the depth of the artery to be punctured, by attaching the directing member such that is adjustable to increase or decrease the depth angle of penetration. This adjustment direction can be defined as being in a plane 16 perpendicular to the imaging plane 11.

To inflate the balloons in a REBOA procedure, syringes prefilled with sterile saline may preferably be provided in the guidance assembly. These syringes may be pre-marked for inflation of the first or second balloon. A flow switch and a pressure gauge may be attached to the nozzle of the each syringe. As an alternative, syringes prefilled with sterile fluid and micro-bubbles may be used. By inflating the balloons with micro-bubbles they become more visible for ultrasound scanning. Thus, the post-REBOA procedure position of the balloons within the aorta can be more easily controlled with ultrasound scanning if the balloons have been inflated with micro-bubbles.

A method of use of the guidance assembly and a double balloon REBOA catheter is described below and illustrated in FIGS. 7a-7c. A guidance assembly is placed in the groin area of the patient, as described above. Preferably the assembly is immobilized on a patient's thigh by means of e.g. Velcro, straps, adhesive or a combination thereof. The case is opened (FIG. 7b) and the guidance device will be located over the groin as described in connection to FIGS. 2-3 above. Preferably, sterile drapes 31, 41 (not shown in FIGS. 7a-7c, but as shown in FIGS. 5 and 6a) are folded out to create a larger sterile field surrounding the case, covering the upper thighs and lower abdomen of the patient.

Thereafter an ultrasound probe 10 is placed in the guidance device 1, 51. In the case where the available ultrasonography probe does not fit the bracket of the guidance device, a probe adapter may be used, to optimally arrange the obtained imaging plane perpendicularly in relation to the directing member, as described previously. If a probe cover is provided, such is pulled or rolled up over the probe. The groin is scanned with the ultrasonography probe held perpendicular to the groin by the guidance device 1, 51, and thus perpendicular to the anticipated course of the common femoral artery. Once the artery is identified, the position of the probe is adjusted until the artery appears in the middle of the image as a circular structure. Depending on the distance between the skin surface and the artery the angle for insertion of the needle is selected and the depth of needle estimated. A needle is inserted through the directing member 2, 52. A chamber or reservoir to collect the blood (not shown) that comes out of the needle is attached to the hub of a needle of suitable diameter and length. The needle is inserted through the skin and slowly advanced until its tip appears on the image on the surface of the artery. The needle is advanced into the artery. Once there is a brisk flow of blood from the needle, the guidance device is separated from the needle. At this stage, if using a removable directing member or a directing member with an open structure, the guidance device may be removed from the needle and the patient's groin area, leaving the needle inserted into the blood vessel. This allows better access to the area by the user. At this stage the ultrasound imaging is not needed anymore.

Using the well-known Seldinger technique, an introducer is placed in the femoral artery. Hence, under ultrasound guidance, as described above, a needle is inserted through the directing member 2, 52, to create a skin puncture. Thereafter an introducer can be inserted through the puncture and into the femoral artery. If the back flow from the needle is not satisfactory, the reservoir may be removed and a pressure transducer coupled to the needle to confirm that its tip is in the artery.

In the case of a REBOA procedure. the occlusion device, with the occlusion device guide in situ, is advanced through the introducer, until as seen using the depth markers on the occlusion device shaft, the occlusion balloon is within the aorta. The balloon is slowly inflated, while the pulse in the opposite groin is felt. Loss of the pulse confirms that the catheter is correctly located.

As outlined above, during REBOA, the balloon catheter will be non-surgically introduced under ultrasound guidance into the femoral artery in one of the groins through a tiny opening in the skin (FIG. 7c). The catheter is advanced until the abdomen balloon, i.e. the proximal balloon, reaches the aortic bifurcation. The cable is connected to a pressure monitor. The abdomen balloon is thereafter inflated using a pre-filled syringe, stopping blood flow to the pelvis. If hemodynamic response is not satisfactory, indicating that the site of bleeding is in the upper abdomen, the thorax balloon, i.e. distal balloon, will be inflated. Based on the inventors experience and that reported in literature, these few steps are sufficient to practically eliminate the risk of catastrophic fall in blood pressure and thereby allow enough time for transport to a hospital. This is true even in patients in whom the source of bleeding is not in the abdomen, as stopping blood flow to the latter leads to redistribution of blood to more vital organs such as the brain and the heart.

All the parts needed to perform the REBOA procedure are preferably contained in the guidance assembly. In FIGS. 6a and 6b, another guidance assembly is illustrated. FIG. 6a shows the guidance assembly 40 in an unfolded state, ready for use, with sterile drapes 41 extending from all sides. FIG. 6b shows the same guidance assembly as in FIG. 6a, but without showing the extending drapes 41, in order to more clearly show the contents. The assembly may be packaged e.g. in a rectangular box measuring about 70×300×400 mm. At the upper left quadrant of the box, sides may be made of a flexible material, while the bottom is flexible, transparent and easy to puncture with a needle. Notably, “upper” herein is meant to be the direction of a patient's head, when the device is in use as described. The rest of the bottom and the sides of the box are preferably made of a semi-stiff absorbent material that keeps its form during transport.

Along at least the upper border of all sides, a sterile drape 41 may be attached. By unfolding the drape the sterile working area is enlarged. At the upper left quadrant or other suitable position, a sleeve or probe cover that can accommodate an ultrasonography probe will be attached to the inner surface of the drape. Using such a sleeve ensures that a (non-sterile) ultrasound probe can be used without contaminating the sterile field of the assembly.

The guidance assembly 40 shown in FIGS. 6a and 6b further comprises a guidance device 1 as described above, as well as an introducer and/or needle for puncture of the skin, a dilator to widen the entry hole, an occlusion device as previously described and preferably two-prefilled syringes for inflating the balloons. All components are releasably attached to the bottom of the case to prevent displacement during transportation. Other accessories such as at least a pair of sterile gloves, sterile ultrasound gel and sterile wipes or sterile fluid can be provided in the assembly.

Preferably, an introducer for use with the guidance device as described herein is provided with a reinforced tip. Thus, it can be placed without the need for incising the skin, eliminating one or more steps in the procedure.

The present invention is not limited to the above-described preferred embodiments. Various alternatives, modifications and equivalents may be used. Therefore, the above embodiments should not be taken as limiting the scope of the invention, which is defined by the appending claims.

1-17. (canceled)

18. A guidance device for ultrasonographic guidance of percutaneous placement of a trocar or an introducer in a subject,
said guidance device being adapted for placement against the body of the subject essentially at a desired percutaneous insertion site for said trocar or introducer, and
wherein said guidance device comprises a directing member configured to receive a trocar or an introducer for percutaneous insertion of said trocar or introducer into a blood vessel of a subject,
wherein said directing member is oriented such that said trocar or introducer received in said directing member provides for insertion of said trocar or introducer along a first axis A,
said guidance device being adapted to receive and releasably hold an ultrasonographic imaging probe to obtain an image having an imaging plane being a first plane,
wherein said guidance device is arranged to hold said ultrasonographic imaging probe such that said first axis A of said directing member is positioned in a second plane that is essentially perpendicular to and transversely crossing said first plane, and
said directing member is coupled to said guidance device via an attachment member such that said directing member is configured to be movable only in said second plane.

19. A guidance device according to claim 18, wherein said guidance device further comprises attachment means for attaching said guidance device on said subject essentially at said desired percutaneous insertion site for said introducer.

20. A guidance device according to claim 18, wherein said guidance device comprises a cavity arranged to hold said probe such that the imaging plane of said probe is arranged essentially perpendicularly to the first axis A of the directing member.

21. A guidance device according to claim 18, wherein an outer surface being adapted for placement against the body of the subject is shaped to correspond to a general shape of a human or mammalian groin area.

22. A guidance device according to claim 18, further comprising a support adapted to correspond to a general shape of a human or mammalian groin area.

23. A guidance device according to claim 22, further comprising a support adapted to correspond to a general shape of a human or mammalian wrist or neck area.

24. A guidance device according to claim 18, wherein said attachment member is a hinge.

25. A guidance device according to claim 19, wherein said attachment means for attaching said guidance device on said subject comprises tape or adhesive.

26. A guidance device according to claim 18, wherein said directing member is removable at said attachment member.

27. A guidance device according to claim 20, further comprising an adapter configured to be arranged in said cavity and receive a distal end of said ultrasonographic imaging probe, said adapter further being configured to hold said probe in a position such that said first axis A of said directing member is positioned in a second plane that is essentially perpendicular to and transversely crossing said first plane.

28. A guidance assembly for ultrasonographic guidance of percutaneous placement of a trocar or an introducer in a subject, comprising:
an integrated guidance device for ultrasonographic guidance,
said guidance device being adapted for placement against the body of the subject essentially at a desired percutaneous insertion site for said trocar or introducer, and
wherein said guidance device comprises a directing member configured to receive a trocar or an introducer for percutaneous insertion of said trocar or introducer into a blood vessel of a subject,
wherein said directing member is oriented such that said trocar or introducer received in said directing member provides for insertion of said trocar or introducer along a first axis A,
said guidance device being adapted to receive and releasably hold an ultrasonographic imaging probe to obtain an image having an imaging plane being a first plane,
wherein said guidance device is arranged to hold said ultrasonographic imaging probe such that said first axis A of said directing member is positioned in a second plane that is essentially perpendicular to and transversely crossing said first plane, and
said directing member is coupled to said guidance device via an attachment member such that said directing member is configured to be movable only in said second plane,
and a container for enclosing and transporting components of said guidance assembly.

29. A guidance assembly according to claim 28, wherein said guidance device comprises a cavity arranged to hold said probe such that the imaging plane of said probe is arranged essentially perpendicularly to the first axis A of the directing member.

30. A guidance assembly according to claim 29, further comprising at least one adapter configured to be arranged in said cavity and receive a distal end of said ultrasonographic imaging probe, said adapter further being configured to hold said probe in a position such that said first axis A of said directing member is positioned in a second plane that is essentially perpendicular to and transversely crossing said first plane.

31. A guidance assembly for performing occlusion of a blood vessel, said guidance assembly comprising:
an occlusion device for occluding a blood vessel of a subject,
an introducer for percutaneous insertion of said occlusion device into a blood vessel of a subject, an integrated guidance device for ultrasonographic guidance,
said guidance device being adapted for placement against the body of the subject essentially at a desired percutaneous insertion site for said trocar or introducer, and
wherein said guidance device comprises a directing member configured to receive a trocar or an introducer for percutaneous insertion of said trocar or introducer into a blood vessel of a subject,
wherein said directing member is oriented such that said trocar or introducer received in said directing member provides for insertion of said trocar or introducer along a first axis A,
said guidance device being adapted to receive and releasably hold an ultrasonographic imaging probe to obtain an image having an imaging plane being a first plane,
wherein said guidance device is arranged to hold said ultrasonographic imaging probe such that said first axis A of said directing member is positioned in a second plane that is essentially perpendicular to and transversely crossing said first plane, and
said directing member is coupled to said guidance device via an attachment member such that said directing member is configured to be movable only in said second plane,
and
a container for enclosing and transporting components of said guidance assembly.

32. A guidance assembly according to claim 31, wherein said occlusion device comprises at least a first balloon for inflation inside a blood vessel, and a second balloon, for inflation inside a blood vessel.

33. A guidance assembly according to claim 28, further comprising sterile drapes adapted to define a sterile field within at least said container.

34. A guidance assembly according to claim 28, further comprising attachment means for attaching said container on said subject essentially at said desired percutaneous insertion site for said trocar or introducer.

35. A method for ultrasonographic guidance of percutaneous placement of a trocar or an introducer in a subject, the method comprising the steps of:
attaching a guidance device according to claim 18 to a subject at a percutaneous insertion site for insertion of a trocar or an introducer,
inserting an ultrasonographic imaging probe in said guidance device,
puncturing said percutaneous insertion site with said trocar or said introducer to access an underlying blood vessel under guidance of an image produced by said ultrasonographic imaging probe,
inserting an introducer through the directing member of the guidance device into said percutaneous insertion site and into said blood vessel under guidance of an image produced by said ultrasonographic imaging probe.

36. A method for ultrasonographic guidance of percutaneous placement of a trocar or an introducer in a subject, the method comprising the steps of:
attaching a guidance assembly according to claim 28 to a subject at a percutaneous insertion site for insertion of a trocar or an introducer,
inserting an ultrasonographic imaging probe in said guidance device,
puncturing said percutaneous insertion site with said trocar or said introducer to access an underlying blood vessel under guidance of an image produced by said ultrasonographic imaging probe,
inserting an introducer through the directing member of the guidance device into said percutaneous insertion site and into said blood vessel under guidance of an image produced by said ultrasonographic imaging probe.

37. A method for ultrasonographic guidance of percutaneous placement of a trocar or an introducer in a subject, the method comprising the steps of:
attaching a guidance assembly according to claim 31 to a subject at a percutaneous insertion site for insertion of a trocar or an introducer,
inserting an ultrasonographic imaging probe in said guidance device,
puncturing said percutaneous insertion site with said trocar or said introducer to access an underlying blood vessel under guidance of an image produced by said ultrasonographic imaging probe,
inserting an introducer through the directing member of the guidance device into said percutaneous insertion site and into said blood vessel under guidance of an image produced by said ultrasonographic imaging probe.
